(19) United States
(12) Patent Application Publication
(10) Pub. No.: US 2007/0048355 A1
(43) Pub. Date: Mar. 1, 2007

(54) Non-irritating solvent-borne polymeric coatings for application to the skin

(76) Inventor: Daniel Perlman, Arlington, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/213,655

(22) Filed: Aug. 26, 2005

(51) Int. Cl. A61K 31/717; A61K 9/70

(52) U.S. Cl. 424/443; 514/57

(57) ABSTRACT

A method and composition for reducing or eliminating stinging that may accompany application of topical solution of a skin-compatible polymeric material to a skin wound, abrasion, burn or other skin injury. The topical solution may contain one or more skin-irritating chemicals, such as diethyl ether, ethanol, ethyl acetate, methyl acetate, acetone, methyl ethyl ketone and combinations thereof. The method includes: (i) providing a composition that contains between 1% and 10% by weight of the skin-compatible polymeric material dissolved in a liquid medium suitable for human topical use in which the liquid medium includes less than 20% combined weight of skin-irritating chemicals, and at least 45% combined weight of at least one volatile solvent and/or volatile co-solvent, in which the molecules constituting this 45% combined weight portion of the liquid medium contain at least 5 carbon atoms, and the liquid medium has an initial boiling point at 1 atmosphere of greater than 35° C. and less than 150° C., and causes little or no stinging when applied to a skin injury, and (ii) applying the composition to the skin of a patient at the site of the skin injury.

### BACKGROUND OF THE INVENTION

This invention relates to improved topical solutions of nitrocellulose and other skin-compatible polymeric materials that are applied as protective coatings, and dried on the human skin, in which the solutions produce little or no stinging or irritation when applied to an abrasion, cut, burn or other skin injury.

Solutions of nitrocellulose (cellulose nitrate) have been used for many years to form protective coatings on the skin. Such solutions are commonly known as collodions, and are described as highly flammable, colorless or yellowish syrupy solutions of pyroxylin (nitrocellulose) dissolved in ether and alcohol. Collodions have been used as adhesives to close small wounds and hold surgical dressings, or in formulating topical medications, or in making photographic plates. Notwithstanding the above definition, for the purposes of the present invention, the definition of the term “collodion” herein is expanded to encompass any solution of nitrocellulose dissolved in a solvent or combination of solvents that may also contain additive ingredients that enhance the quality of the product on the skin. Therefore, a collodion formulated according to the present invention might not include either alcohol or ether, while it may contain plasticizers, emollients, humectants and the like.

When a chemically nitrated cotton (or other form of nitrated cellulose) or an alternative skin-compatible polymeric material is dissolved in a volatile organic solvent, and is applied over a burn or skin wound, the solvent rapidly evaporates leaving a clear flexible protective coating. The Merck Index (Ninth Edition, Merck and Co., Rahway, N.J.) describes simple collodion as a solution of 4 g pyroxylin (chiefly nitrocellulose, i.e., cellulose nitrate) in 100 ml of a mixture of 25 ml alcohol (ethyl alcohol) and 75 ml ether (diethyl ether), that when exposed in thin layers, evaporates leaving a tough colorless film. The same reference describes alternative compositions derived from simple collodion including “flexible collodion” that is formulated by combining 2% by weight camphor and 3% by weight castor oil with simple collodion, and “styptic collodion” that is formulated by combining 18% by weight tannic acid with flexible collodion. The latter composition is reported to contain about 61% ether and about 21% alcohol solvent by volume (Merck Index). A current Material Safety Data Sheet published by Mallinckrodt Baker, Inc. (Phillipsburg, N.J.) similarly states that flexible collodion, U.S.P. contains nitrocellulose (CAS No. 9004-70-0), diethyl ether (CAS No. 60-29-7), ethyl alcohol (CAS No. 64-17-5), camphor (CAS No. 76-22-2) and castor oil (CAS No. 8001-79-4). It is appreciated by those familiar with the art of nitrocellulose, that ether and alcohol act as co-solvents (also known as latent solvents) in the dissolution of nitrocellulose, and that neither chemical alone is a satisfactory nitrocellulose solvent.

A variety of ingredients have been added to collodion solutions formulated for topical application to confer desirable properties to the resulting dried films formed on the skin.

For example, Popp in U.S. Pat. Nos. 5,433,950 and 5,525,358 describes a flexible collodion film-forming composition containing a polymeric agent, polybutylene, a keratolytic agent, salicylic acid, and a crystallization inhibitor including ethyl lactate and castor oil. The resulting film is reported to have improved resilence, durability and resistance to rub-off, while the crystallization inhibitor prevents the salicylic acid from precipitating. Popp also describes the addition of local anesthetics such as benzocaine and lidocaine to the collodion formulation to help alleviate localized discomfort that can accompany multi-day keratolyic treatment of skin warts and the like.

Studin in U.S. Pat. No. 6,337,076 describes a method and composition for treating an adverse skin condition such as hypertrophic scarring based upon applying a liquid collodion film-forming carrier and a dermatologically effective amount of an active ingredient such as a steroid, silicone gel or vitamin to the affected area of skin.

Several companies including Del Pharmaceuticals, Inc. (Farmingdale, N.Y.) and Medtech, Inc. (Jackson, Wyo.) produce topical solutions containing skin-compatible polymeric materials, e.g., nitrocellulose, that form protective coatings on the skin upon solvent evaporation. In the nitrocellulose-containing over-the-counter topical products based upon modified flexible collodion, the ether co-solvent component used in traditional collodion has been replaced with a somewhat less volatile organic solvent such as acetone or ethyl acetate. Another company, Beiersdorf AG (Hamburg, Germany), produces a different evaporative skin coating formulation known as Curad® Spray Bandage based upon a different skin-compatible polymeric material such as an acrylate-containing polymer, e.g., poly(methylacrylate-isobutene-monoisopropylmaleate), a random co-polymer material, that is dissolved in an ethyl acetate-containing solvent.

In spite of the existence of several different liquid bandage formulations containing either nitrocellulose or an alternative skin-compatible polymeric material, many consumers who use these products experience unpleasant stinging when the formulations are applied to an open wound, burn, or skin abrasion. The remedy for this problem has remained elusive. To the best of Applicant's knowledge, it has not been determined whether this stinging is attributable to one or to several components in the formulation. There has been speculation as to whether some pharmacological or chemical agent could be added to these formulations to combat stinging, or alternatively, whether an ingredient could be removed from the formulation to reduce stinging. The present invention involves researching this question and resolving the stinging problem.

### BRIEF SUMMARY OF THE INVENTION

The present invention concerns improved topical formulations that contain skin-compatible polymeric materials such as nitrocellulose or acrylate-containing polymers and co-polymers such as the random co-polymer, poly(methylacrylate-isobutene-monoisopropylmaleate), that are dissolved in volatile organic solvents, in which these evaporative coating formulations are less irritating when applied to the skin for protecting wounds, burns, abrasions, and the like. Such solutions may be used for treating these or other dermatological conditions.

The present invention involves finding both the cause and the remedy for stinging, burning and irritation that accompany application of presently available collodions and other solubilized skin-compatible polymeric materials such as the acrylate-containing polymers and co-polymers, to wounds, abrasions and other skin injuries. More specifically, Applicant has discovered that the stinging or burning sensation commonly experienced on the skin is primarily attributable to the presence of certain low molecular weight solvents, latent solvents (also termed co-solvents herein) and diluents used for dissolving and diluting the skin compatible polymeric material, e.g., nitrocellulose. In particular, the methyl, ethyl, and to a lesser degree, the propyl derivatives among the acetate, alcohol, ketone and ether solvent and co-solvent groups cause such stinging and burning. These low molecular weight solvents have been traditionally utilized because they are volatile and beneficially accelerate the rate of drying of such coatings that are applied to the skin.

Pursuant to the present discovery, the solvent system used to solubilize nitrocellulose and other constituents in evaporative coatings such as collodion has been altered primarily by removing or substantially reducing the level of methyl, ethyl and some propyl derivatives of solvents, co-solvents and diluents formerly present in such liquids. For example, methyl and ethyl acetate solvent concentrations can be reduced or eliminated, while the concentrations of ethyl and isopropyl alcohol co-solvents currently used can be similarly reduced or eliminated. Likewise, currently used lower ketone solvents such as acetone and methyl ethyl ketone can be reduced or eliminated, as can lower ether co-solvents such as diethyl ether. Applicant has substituted or greatly increased the levels of certain solvents, co-solvents and diluents that have been discovered to be completely or substantially “sting-free.” The liquid ingredients are still sufficiently volatile to allow a nitrocellulose coating to dry rapidly, or at very least, to thicken and/or gel rapidly before drying. Examples of non-stinging solvents and diluents include the nitrocellulose solvents, n-propyl acetate, n-butyl acetate, n-amyl acetate, their structural isomers and combinations thereof, and the alkane diluents including n-pentane, n-hexane, n-heptane and n-octane and their structural isomers, and combinations thereof. For example, if n-butyl acetate is substituted for ethyl acetate as the primary nitrocellulose solvent, and an alkane (e.g., hexane or heptane) diluent is used in place of ethanol, isopropanol or ether, the stinging and burning problem is largely eliminated when collodion is applied to wounds, burns, abrasions, irritated skin or other skin injuries.

### DETAILED DESCRIPTION OF THE INVENTION

Definitions

For terms that are not defined below, the common definition is assumed as provided in the current edition of Webster's International Dictionary or alternatively, provided in a standard organic chemistry textbook such as Organic Chemistry (5^{th} Edition) by Leroy Wade (Prentice-Hall, Inc). As used in this description and the accompanying claims, the following terms shall have the meanings indicated, unless the context requires otherwise:

The term “sting,” “stinging,” or “burning” as used herein means the sensory perception or skin sensation of burning, itching, pain or other form of acute discomfort that accompanies topical application of a composition, e.g., a conventional collodion liquid, to the skin. In the context of the present invention, stinging is typically localized to an area of the skin that generally encompasses a wound, abrasion, burn, blister or other skin injury that has been treated with a collodion liquid.

A “skin-irritating chemical” is a chemical that causes “stinging” or “burning” when applied as described above and achieves a rating of “moderate” to “strong” sting in a rating scheme such as is described herein. Exemplary skin-irritating chemicals include diethyl ether, ethanol, ethyl acetate, methyl acetate, acetone and methyl ethyl ketone.

As stated earlier, the term “collodion” as used herein has been expanded for the purposes of the present invention to include any nitrocellulose material or derivative thereof that has been dissolved in any solvent, combination of solvents and/or co-solvents including solvent systems that do not necessarily include either ether or alcohol. Collodion compositions may contain one or more skin-irritating solvents such as diethyl ether, ethanol, ethyl acetate, methyl acetate, acetone, methyl ethyl ketone, and combinations thereof. Collodions may include a host of other additives as well.

The term “nitrocellulose” includes cellulose nitrate and its derivatives. The material is often a pulpy or cotton-like polymer derived from cellulose typically by treating with sulfuric and nitric acids, and used in the manufacture of many materials including, for example, explosives, plastics, microporous membranes, lacquers, and collodion liquid coatings (see above). It is the main film-forming component in the compositions described in the present invention. Nitrocellulose is typically provided in commerce as a moistened material (e.g., 30% by weight isopropanol:70% by weight dry nitrocellulose) to prevent accidental ignition or explosion. However, unless stated otherwise, percentage weights of nitrocellulose specified in compositions herein are expressed as dry weight percentages.

When nitrocellulose is solubilized, i.e., dissolved, in any solvent system such as a solvent blend including n-butyl acetate and n-hexane, the resulting solution is termed “collodion.” Unlike earlier definitions of collodion, the definition of collodion herein does not require the presence of either alcohol or ether solvent. Collodions as defined herein may also contain a variety of additives that enhance topical use of the product.

The term “skin-compatible polymeric material” refers to nitrocellulose as well as any other polymeric material such as the acrylate polymers and co-polymers (e.g., poly(methylacrylate-isobutene-monoisopropylmaleate), that are safe for topical application to the skin under FDA regulations and guidelines, that can be dissolved in a solvent system compatible with forming a coherent evaporative coating that is preferably strong, flexible, adherent, water-resistant and breathable when dried onto the skin.

The term “acrylate polymer” (or co-polymer) refers to any polymer or co-polymer material assembled using acrylic acid, acrylic acid derivatives, e.g., methyl acrylate, ethyl acrylate, and other monomer reactants (not necessarily acrylate moieties) to generate polymer and co-polymer molecules. In the case of co-polymers containing acrylates and other moieties, monomer units may be assembled either randomly, e.g., poly(methylacrylate-isobutene-monoisopropylmaleate, or non-randomly to produce polymers having different physical characteristics.

If a nitrocellulose or other polymer or co-polymer-containing solution or other liquid medium is referred to as “topical” or “suitable for topical use,” these terms mean that the composition is safe for application to the skin as defined by current FDA regulations.

The term “plasticizer” refers to a chemical agent included in the liquid coatings described herein, that adds simple flexibility or plasticity to the otherwise brittle nitrocellulose-based films once they have dried on the skin as described herein. A plasticizer such as castor oil may have a dual function, providing some beneficial emollient properties to the skin as well. Plasticizers can also add bulk to a film and improve the uniformity, appearance and/or strength of the film without substantially increasing the viscosity of the liquid coating material used to form the film.

The term “evaporative liquid composition” or “evaporative coating” refers to a liquid coating in which non-volatile components dissolved or dispersed in the liquid, form a solid film by simple evaporation of the solvents present in the liquid after the liquid has been applied to the skin.

The term “solvent”, “volatile solvent”, “nitrocellulose solvent” as used herein means a liquid chemical such as butyl acetate, (an alkyl acetate) or propyl acetate that is capable of dissolving nitrocellulose at room temperature, and that evaporates with sufficient rapidity that when coated onto human skin (e.g., using a small brush) at a temperature of 37° C., most of the liquid, i.e., 80% or more, will evaporate within approximately five minutes and preferably within 2 minutes or less. The “volatile alkyl (or hydrocarbon) acetate solvents” used herein are usually selected from the group consisting of n-propyl acetate, n-butyl acetate, n-amyl acetate, structural isomers of n-propyl, n-butyl acetate and n-amyl acetate (such as tert-butyl acetate) and combinations thereof.

Referring to the number of carbon atoms in solvent molecules such as the alkyl acetates for example, propyl acetate contains 5 carbon atoms and butyl acetate contains 6. With diluent liquids such as alkane solvents, pentane for example, contains 5 carbon atoms, hexane contains 6, and heptane contains seven.

The term “isohexane” refers to one or more structural isomers of hexane, other than n-hexane, and may include one or more of the following: 2-methylpentane; 3-methylpentane; 2,2-dimethylbutane; or 2,3-dimethylbutane.

The term “initial boiling point” as it refers to a solvent, diluent or a mixed liquid medium means the temperature at which the liquid first starts boiling, given an external pressure of 1 atmosphere (760 millimeters of mercury).

The terms “latent solvent” and “co-solvent,” with or without the prefix word “volatile,” are synonyms as used herein. Furthermore, the singular term “co-solvent” refers to one or to multiple molecular species of co-solvent. The terms refer to one or more organic liquids such as alcohols that can partially dissolve a polymeric material such as nitrocellulose when used alone, or fully dissolve the material in combination with certain other co-solvents (e.g., an alcohol combined with an ether). While the lower molecular weight alcohols such as ethanol and isopropanol contribute to stinging, the higher molecular weight alcohols such as butyl alcohol, amyl alcohol and their structural isomers cause less stinging and may be useful. Co-solvents should be sufficiently volatile to evaporate at a rate comparable to, or faster than the solvent(s) (see above) to accelerate the thickening, gelling, and immobilization of a polymeric coating on the skin.

The terms “diluent” and “non-solvent” are synonyms as used herein. The terms refer to an organic liquid such as an alkane, e.g., pentane, hexane or heptane, that does not solubilize nitrocellulose, but is miscible with a true solvent for nitrocellulose such as butyl acetate, and can be used as an effective diluent or thinner for a nitrocellulose solution that also includes at least one true solvent. A diluent or non-solvent, when used in the present invention, should evaporate at a rate at least comparable to, or faster than the solvent and/or co-solvent. Other organic liquids including other hydrocarbons such as toluene can serve as diluents but are less preferred due to toxicity considerations.

The term “volatile aliphatic (or hydrocarbon) diluent” refers to an evaporating diluent liquid such as a 5, 6, 7 or 8 carbon-containing straight or branched chain alkane (pentane, hexane, heptane or octane) or mixture of such alkanes and/or their structural isomers that are miscible with the nitrocellulose solvent, and causes little or no stinging when applied to a skin injury. This diluent liquid should evaporate more rapidly on the skin than the nitrocellulose solvent (see “solvent” above) and preferably has an initial boiling point at 1 atmosphere of greater than 35° C. and preferably less than 120° C.

The phrase “little or no stinging” means little or no sensation of pain, burning or other discomfort following application of a collodion liquid or other test substance to a wound, blister, burn, abrasion or otherwise injured portion of skin. Notwithstanding this definition, it is generally appreciated that different individuals perceive pain to different extents. Therefore, some variation is to be expected among individuals who score different solvents for their degree of sting. According to the scoring system described above, little or no stinging would be scored by individuals as between a single plus (+) and a minus (−).

The term “reducing or eliminating stinging” as it refers to a collodion composition that is applied to the skin, means that a collodion liquid that may have originally caused substantial stinging, e.g., scored as (+++) is improved to a score of either (+) or even (−). While the improved collodion liquid remains an organic solvent-based nitrocellulose solution, the skin-irritating solvents (including diethyl ether, ethanol, ethyl acetate, methyl acetate, acetone, methyl ethyl ketone and combinations thereof) are largely eliminated or greatly reduced in abundance. Instead, nitrocellulose solvents such as the alkyl acetate solvents are selected including those from the group consisting of n-propyl acetate, n-butyl acetate, n-amyl acetate, structural isomers of n-propyl acetate, n-butyl acetate and n-amyl acetate, and combinations thereof.

Examples of collodion liquids that may be modified with regard to their solvent compositions include simple collodion, flexible collodion, styptic collodion, cantharidal collodion, and any other solvent-solubilized nitrocellulosic material in which the liquid medium is optionally supplemented with at least one topically active pharmacological agent. Definitions for these individual collodions may be found in the Merck Index or via a database web search (e.g., www.google.com).

The term “topically active pharmacological agent” as used herein means a chemical or biochemical agent that, under FDA regulations, is suitable for application to the skin in a nitrocellulose-based coating. These include suitable hormones, local anesthetics, keratolytic agents, ultraviolet light protective agents, and combinations thereof.

The term “humectant” as used herein refers to a substance or agent suitable for topical use that promotes retention of skin moisture, e.g., methyl propanediol.

The term “emollient” as used herein refers to a substance or agent suitable for topical use that helps maintain skin suppleness, and may also be soothing, e.g., castor oil or other triglyceride mixture.

The term “antiseptic” as used herein refers to a substance or agent suitable for topical use (i.e., such as benzalkonium chloride approved by the FDA) that prevents or arrests the growth of microorganisms (e.g., bacteria, viruses and molds) in a skin wound, burn, or other skin injury.

When Applicant commenced research to remedy the collodion stinging problem, it was believed that organic solvents in general caused the problem. Since organic solvents are needed for dissolving nitrocellulose, they could not be eliminated. Therefore, the direct approach to solving this problem seemed to require finding an analgesic agent to counteract the sting. After many analgesics were tested and failed to substantially decrease stinging, Applicant considered the possibility that differences in “stinging intensity” might exist among different nitrocellulose solvents. If such differences existed, they might lead to an improved formulation. As is evident in experiments described below, very dramatic differences in stinging intensity were observed, even among members of the same chemical family of solvent or co-solvent that were tested (e.g., among the acetate solvents). This finding led to empirical testing of a variety of solvents, co-solvents, diluents, plasticizers and other additives added at different concentrations to the collodion system.

Modification of the nitrocellulose liquid vehicle according to the teaching of the present invention is widely applicable to any and all types of collodion coatings used on the skin, e.g., flexible collodion, pharmacologically supplemented collodions, and other nitrocellulose-based coatings containing a variety of solubilized components. For the purpose of the present invention, nitrocellulose-based coatings for application to human or other animal skin are hereinafter collectively referred to as either nitrocellulose or “collodion” solutions or coatings regardless of the solvent system selected.

As suggested above, the motivation for this invention stems from the experience and appreciation that collodion liquid coatings, as well as non-nitrocellulose skin-compatible polymeric coating liquids, typically cause stinging and sometimes inflammation when applied to injured skin. For example, Applicant obtained two commercial liquid bandage collodion products including: (i) New Skin™ that is described by the manufacturer (Medtech, Inc., Jackson, Wyo.) as a first aid antiseptic liquid bandage, and (ii) Skin Shield™ produced by Del Pharmaceuticals, Inc. (Farmingdale, N.Y.). Both of these products caused stinging when applied to abraded skin. Skin Shield™ contains dyclonine hydrochloride (a topical anesthetic), benzethonium chloride (an antiseptic), acetone, amyl acetate, castor oil, drometrizole, nitrocellulose, and denatured alcohol. On the other hand, New Skin™ contains pyroxylin solution (nitrocellulose), denatured alcohol, oil of cloves, 8-hydroxyquinoline (antiseptic), ethyl acetate, butyl acetate and amyl acetate in addition to some other minor ingredients. A third liquid bandage product known as Curad® Spray Bandage produced by Beiersdorf AG (Hamburg, Germany) also causes stinging when applied to injured skin. Instead of nitrocellulose, it contains the skin-compatible polymeric material, poly(methylacrylate-isobutene-monoisopropylmaleate) dissolved in a liquid medium containing ethyl acetate among other ingredients.

Considering the various boiling points of the different solvents (ethyl acetate 77° C., acetone 57° C. and diethyl ether 35° C.), it is likely that the selection of ethyl acetate in two of the above products (over acetone and ether) provides a somewhat slower rate of evaporation and therefore better control over drying of the film on the skin. In fact, excessively rapid evaporation of solvents including ether and/or acetone that have been used in collodion products can cause rapid cooling and ambient water vapor condensation (also known as “blushing”) that may harm the integrity of the collodion film.

In approaching the problem of stinging and irritation that accompanies application, of collodion to a skin injury such as a burn, cut or abrasion, Applicant searched the patent literature and academic literature for agents that might be added to collodion to alleviate the problem.

Popp, for example, in U.S. Pat. Nos. 5,433,950 and 5,525,358 adds topical anesthetics such as benzocaine and lidocaine to collodion to control long term discomfort associated with salicylic acid keratolytic treatment of the skin. Similarly, the general literature on topical anesthetics suggests that addition of certain alcohol and ketone compounds including benzyl alcohol, camphor, menthol, phenol, resorcinol and combinations thereof might be helpful if added to collodion solutions to reduce stinging.

On the other hand, Hahn et al. in U.S. Pat. No. 5,756,107 has identified water-soluble salts of lithium and potassium cations that possess anti-irritant activities, and these agents can be added to topical vehicles for application to the skin. It is claimed that these agents can reduce or eliminate skin irritation caused by chemical agents applied to the skin such as exfoliants, sunscreens, shaving products and soaps, for example. Similarly, Hahn et al. in U.S. Pat. No. 5,716,625 identifies aqueous soluble salts of strontium that inhibit skin irritation. Other investigators have shown that the anti-inflammatory properties of at least strontium and selenium cations appears to be mediated through controlling the appearance of keratinocyte-derived inflammatory cytokines.

Given the above published literature on controlling pain and inflammation of the skin using topically applied agents, Applicant wished to determine whether any of the above anti-irritants would measurably reduce the stinging caused by collodions. As a first step, a testing procedure was established as described below.

Sting Testing Procedure

The preparations described above were applied to skin that had been carefully abraided shortly before testing to determine whether the stinging and discomfort caused by collodion application could be suppressed by one or more of these agents. The procedure used was to manually scour a finger knuckle using 100 grit garnet sandpaper just to the point of slight bleeding, and then allowing the skin to rest a minute or so until any discomfort, i.e., sensation of irritation, pain or heat, had disappeared prior to challenging the abrasion with a potential chemical irritant. The resultant stinging sensation was compared to that produced with the unmodified commercial New Skin™ collodion product (the “control”). Sting (also termed and described as “burning sensation”) was rated on a five level scale as follows: (−) no sting, (+/−) barely detectable sting, (+) slight sting, (++) moderate sting, (+++) strong sting. The unmodified control sample of New Skin™ registered +++. In these tests, Applicant typically detected the stinging sensation from the collodion within between 3 and 15 seconds after applying each sample. The sting soon diminished and usually faded away within a minute or so after applying the collodion. This method of testing and scoring the level or magnitude of the stinging sensation is referred to herein as the “sting test” or “standard sting test.”

Anti-Irritant Agents Added to Collodion do not Block Stinging Sensation

Specified amounts of several of the above analgesic agents were added and dispersed into individual one gram samples of a commercial collodion product (New Skin™ Liquid Bandage; Medtech, Inc.). More specifically, to individual 1 gram samples of this liquid collodion were added:

(a) between 25 mM and 100 mM of lithium acetate dihydrate,

(b) between 2% and 4% by weight phenol,

(c) between 2% and 10% by weight benzyl alcohol,

(d) between 1% and 2% by weight lidocaine,

(e) between 5% and 10% by weight benzocaine, and

(f) between 2% and 4% by weight methyl propanediol (a skin humectant)

As an experimental “control” and sensory reference for a +++ level of stinging, a sample of New Skin™ collodion without any additive was always skin-tested alongside the other samples.

To maximize the solubility of lithium acetate in (a) above, while also minimizing the tendency of collodion to desiccate the skin, 2.5% by weight water was added to the collodion before adding the salt. This concentration of water was found to remain soluble in the collodion liquid. As an additional control, a sample of the original collodion containing 2.5% water but without the lithium salt was also tested.

Surprisingly, all of the supplemented collodion samples [(a) through (f)] also registered +++ indicating that neither the anesthetics nor analgesic agents provided any measurable relief from the stinging of the collodion. Nor did the addition of humectant or 2.5% water decrease the stinging caused by the collodion. In fact, the pain sensation was perceived as being increased by addition of water. This surprising observation ran counter to a previous hypothesis of Applicant, i.e., that stinging might be caused by loss of moisture or desiccation of exposed tissue and nerve endings in a skin wound.

It is believed that the anesthetic effect of various agents tested, e.g., lidocaine, benzyl alcohol and lithium salts, all require substantially longer periods of time to act, i.e., minutes rather than several seconds. Therefore, the stinging caused by the collodion composition could not be blocked by any of these agents. It is likely that if an anesthetic agent such as lidocaine were applied to the skin several minutes before the collodion liquid, stinging could be prevented. However, a two-step process for collodion application was deemed impractical.

Testing Individual Solvents and Other Components in Collodion for Stinging Sensation

Applicant has observed that the stinging sensation is felt very quickly after applying collodion to injured skin, i.e., typically within several seconds. Therefore, it may be difficult or impossible to block this pain sensation by simultaneous addition of any topical analgesic agent. As an alternative approach, the collodion solution might be modified by finding a new combination of chemicals that would not cause stinging, or that could be used at moderate levels that would not cause stinging. To begin with, Applicant individually “sting-tested” the principal liquid ingredients present in the currently formulated New Skin™ Liquid Bandage product. The reasoning used by Applicant was that if the sting sensation for the whole formulation were roughly equal to the sum of its parts, then a series of individual ingredients that did not sting might be combined into a whole that also did not sting.

Using the “standard sting test” described above, and similarly abraded skin on a finger knuckle, individual collodion ingredients were tested neat (undiluted). Accordingly, a droplet or two of the mixture or the chemical ingredient was placed on freshly abraided skin. The results were as follows:

| | INGREDIENT | LEVEL OF STING |
|---|---|---|
| 1. | Commercial New Skin ™ formulation (control) | +++ |
| 2. | Ethyl Alcohol | +++ |
| 3. | Isopropyl Alcohol | ++ |
| 4. | Ethyl Acetate | +++ |
| 5. | n-Butyl Acetate | +/− |
| 6. | Amyl Acetate (mixed isomers) | +/− |
| 7. | Castor Oil | − |

Observations and Conclusions: These dramatic results indicate great differences in stinging potency among the ingredients. The ethyl acetate solvent and ethyl alcohol co-solvent appear to be implicated in the stinging problem since both are present at substantial levels in the product. The preliminary conclusion is that both ingredients should be reduced or replaced in order to beneficially reduce overall sting. Since these highly volatile solvents also enable the collodion coating to dry rapidly on the skin, one or more alternative volatile solvents should be found as replacements. Selection of alternative solvents and co-solvents should be based upon their ability to dissolve nitrocellulose, substantial volatility, low human toxicity (ingestion, breathing, skin sensitivity), chemical compatibility with other ingredients, and solubility in the overall formulation.
Additional Testing of Ingredients

Various non-aqueous solvents can be used to dissolve nitrocellulose. Some of these include true solvents for nitrocellulose including esters/acetates (e.g., ethyl acetate), ketones (e.g., acetone), glycol ethers (ethyl glycol ether), cyclic ethers (propylene carbonate), and lactones (butyrolactone). Other solvents are actually only co-solvents or latent solvents and dissolve nitrocellulose when combined with true solvents or occasionally other co-solvents. These include alcohols and ethers. For example, ethyl alcohol and diethyl ether in traditional collodion act together as two co-solvents to dissolve nitrocellulose. When combined with nitrocellulose, many other organic liquids are actually non-solvents for nitrocellulose, but may be miscible with true solvents, and are therefore termed “diluents” for nitrocellulose solutions. Most potential diluents that Applicant has tested failed to be useful in the present invention for any one of a variety of reasons including toxicity, unsuitable rates of evaporation or contributing to the stinging problem. Considering the volatile alkane hydrocarbon diluents that Applicant has tested, pentanes evaporate very rapidly (n-pentane boiling point=36° C.), while octanes evaporate somewhat slowly (e.g., n-octane boiling point=125° C.). N-hexane, n-heptane and their structural isomers with boiling points between approximately 60° C. and 100° C. turned out to be particularly useful as diluents when combined with true solvents in formulating brush-on applied coatings. For spray-on applications requiring rapid drying of the applied coating, a rapidly evaporating diluent such as n-pentane may be a useful constituent.

Several new chemicals and solvents of potential interest were tested, and others that were formerly tested (all in their neat state) were re-tested using the standard sting test procedure and accompanying scoring system described above. Applicant's strategy behind this testing was to find a true solvent or alternatively, a combination of true solvent and either co-solvent or diluent, that would have sufficient volatility to allow rapid drying of a collodion coating without causing stinging when applied to the skin. The following results were obtained:

| | INGREDIENT | LEVEL OF STING |
|---|---|---|
| 1. | New Skin ™ commercial collodion (control) | +++ |
| 2. | Methyl acetate | +++ |
| 3. | Ethyl acetate | +++ |
| 4. | n-Propyl acetate | + |
| 5. | Isopropyl acetate | + |
| 6. | n-Butyl acetate | +/− |
| 7. | tert-Butyl acetate | +/− |
| 8. | amyl acetate (mixed isomers) | +/− |
| 9. | Acetone | +++ |
| 10. | Methyl ethyl ketone | +++ |
| 11. | Methyl propyl ketone | ++ |
| 12. | Methyl alcohol | +++ |
| 13. | Ethyl alcohol | +++ |
| 14. | Isopropyl alcohol | ++ |
| 15. | n-butyl alcohol | ++/+ |
| 16. | isobutyl alcohol | + |
| 17. | n-amyl alcohol | + |
| 18. | n-Hexane | − |
| 19. | Isohexane (e.g., 2-methyl pentane | − |
| | and other isomers) | |
| 20. | n-Heptane | − |
| 21. | n-Octane | − |
| 22. | Methyl propanediol | + |
| 23. | Castor oil | − |

Observations and Conclusions: Many of the alcohols, ketones, and acetates that were tested caused stinging when applied to compromised or injured skin. Surprisingly, the higher molecular weight solvent species with greater numbers of carbon atoms in each group caused less stinging while the lower molecular weight members of these three groups (e.g., the methyl and ethyl derivatives) produced considerably more stinging. More specifically, the 5, 6 and 7 carbon acetates, propyl acetate, butyl acetate, amyl acetate and their isomers caused much less stinging than the 3 and 4 carbon acetates, methyl acetate and ethyl acetate. The 5 carbon alcohol, n-amyl alcohol caused less stinging than methanol, ethanol and isopropanol. The 5 carbon ketone, 2-pentanone (methyl propyl ketone) caused less stinging than the 4 and 3 carbon ketones, methyl ethyl ketone and acetone.

Interestingly and surprisingly, none of the aliphatic hydrocarbon diluent solvents that were tested caused any stinging. These included a number of alkanes, i.e., n-pentane, n-hexane, n-heptane, n-octane and their isomers having boiling points above 35° C. and having 5, 6, 7 and 8 carbon atoms per molecule. N-pentane with 5 carbon atoms has a low a boiling point (36° C.), and may be more useful in spray-applied coatings requiring rapid drying, than in brush-on coatings. The “no-sting” result is particularly surprising with hydrocarbon diluents such as the alkanes because they tend to dry out and remove natural fats and oils from the skin, and could have proven to be irritating. The ability to use a simple aliphatic solvent such as hexane as a miscible and volatile diluent to thin nitrocellulose in an acetate solvent system such as butyl acetate is particularly valuable. More specifically, alkanes can be selected that will be more volatile than the acetate solvent, and therefore beneficially accelerate the process of thickening, immobilization and drying of the nitrocellulose film on the skin. In effect, a rapidly evaporating aliphatic diluent such as pentane, hexane, heptane or isomers thereof, can replace other rapidly evaporating but less desirable solvents and co-solvents such as ethyl acetate and ethanol that have now been shown to cause stinging or burning when applied to injured skin.

The neurophysiology that underlies these surprising differences in degree of solvent-induced sting, i.e., sensory nerve depolarization, remains to be determined. There is evidence herein to indicate that non-polar solvents such as the alkanes cause considerably less stimulation of pain receptors than polar solvents (including the alcohols, ketones and acetates), while larger polar molecules such as butyl and amyl acetate cause less pain receptor stimulation than smaller polar molecules such as methyl and ethyl acetate.

The above experiment provided the rationale for altering collodion compositions by removing or at least reducing the usage concentration of lower alcohols, ethers, acetates, and ketones including ethanol, isopropanol, ethyl ether, methyl and ethyl acetate, acetone and methyl-ethyl ketone among others. The higher acetate solvents, e.g., butyl acetate, amyl acetate, possibly propyl acetate and their isomers, proved to be useful nitrocellulose solvents, and the aliphatic solvents such as hexane, heptane (and for faster and slower evaporation applications, pentane and octane respectively) and their isomers proved to be useful diluents or “non-solvents” that could be combined with actual nitrocellulose solvents. As the molecular weight of a solvent type (e.g., alkyl acetate) increases, its vapor pressure and rate of evaporation decreases. Thus, alkyl acetates and alkanes whose molecules have more than 7 or 8 carbon atoms tend to evaporate too slowly to be preferred as the principal constituents in the formulations described herein. In addition, when a diluent or latent solvent such as hexane, heptane or isobutyl alcohol is included, it preferably possesses an evaporation rate that is more rapid than the true solvent(s), e.g., n-butyl acetate, that is also present in the formulation. Attention to evaporation rates helps avoid film-damaging nitrocellulose precipitation or flocculation that would be expected to occur if the solvent evaporated first, leaving a non-solvent environment. That is, as the nitrocellulose coating undergoes reduction and drying, it is essential that the last liquid to evaporate from the coating is a solvent rather than a diluent so that the homogeneity and integrity of the nascent film is preserved.

Thus, in a first aspect, the invention features a method of reducing or eliminating stinging that may accompany application of a topical solution of a skin-compatible polymeric material to a skin wound, abrasion, burn or other skin injury. The solution may contain one or more skin-irritating chemicals, such as diethyl ether, ethanol, ethyl acetate, methyl acetate, acetone, methyl ethyl ketone and combinations thereof. The method includes: (i) providing a composition containing between 1% and 10% by (dry) weight of the skin-compatible polymeric material dissolved in a liquid medium suitable for human topical use, in which the liquid medium contains less than 20% (combined weight percent) of one or more skin-irritating chemicals such as the above, and at least 45% (combined weight percent) of at least one volatile solvent and/or volatile co-solvent, in which the molecules constituting the stated 45% solvent and/or co-solvent contain at least 5 carbon atoms. Furthermore, the liquid medium has an initial boiling point at 1 atmosphere of greater than 35° C. and less than 150° C., and causes little or no stinging when applied to a skin injury, and (ii) applying the above-described composition to the skin of a patient at the site of the skin injury.

While an upper limit of 20% by weight is placed upon the amount of skin-irritating chemicals, e.g., ethyl acetate solvent or acetone, that may be included in compositions of the present invention, it is preferred that considerably less, e.g., 10%, 5% or even 0% by weight of skin-irritating solvents be included in compositions of the present invention. The presence of 1%-5% of some skin-irritating chemicals often causes little or no problem. For example, nitrocellulose is typically moistened with 30% by weight alcohol (e.g., isopropanol) during production and shipping to prevent risk of explosion. When, for example, 5%-7% of this moistened nitrocellulose is used in a composition of the present invention, approximately 2% alcohol is passively added. The resulting compositions perform satisfactorily in sensory testing for sting.

While a lower limit of 45% by weight is placed upon the combined amount of volatile solvent(s) and/or volatile co-solvent(s), it is preferred that considerably more, e.g., 55%, 65%, 75%, 85% or even greater amounts of these substances be included in compositions of the present invention. However, it is often desirable to include one or more non-solvents (also termed diluents) in these compositions, in which the proportion of diluent may approach approximately 40% of the composition by weight or more (see hexane diluent in Example 2 below). Therefore, setting a modest lower limit for the proportion of solvent plus co-solvent is important in allowing adequate “space” in the formulation for accommodating a diluent that beneficially affects the overall performance of the composition as described herein.

In one embodiment of the above aspect, the skin-compatible polymeric material is nitrocellulose.

In another embodiment of the above aspect, the skin-compatible polymeric material is an acrylate-containing polymer or co-polymer such as poly(methylacrylate-isobutene-monoisopropylmaleate.

In still another embodiment, at least one volatile solvent in the composition is an acetate solvent.

In a related embodiment, the acetate solvent is an alkyl acetate solvent selected from the group consisting of n-propyl acetate, n-butyl acetate, n-amyl acetate, structural isomers of n-propyl acetate, n-butyl acetate and n-amyl acetate, and combinations thereof.

In another related embodiment, the acetate solvent is an alkyl acetate solvent selected from the group consisting of n-butyl acetate, n-amyl acetate, structural isomers of n-butyl acetate and n-amyl acetate, and combinations thereof.

In another embodiment, the liquid medium is a nitrocellulose solution composition selected from the group consisting of collodion, flexible collodion, styptic collodion, cantharidal collodion, and any other nitrocellulose-based solution composition that is supplemented with at least one topically active pharmacological agent.

In a related embodiment, the pharmacological agent is selected from the group consisting of hormones, local anesthetics, keratolytic agents, ultraviolet light protective agents, and combinations thereof.

In another related embodiment, the hormone is selected from the group consisting of steroids, sexual hormones, and metabolic regulatory hormones.

In another aspect, the invention features a method of reducing or eliminating stinging that may accompany application of a topical solution containing a skin-compatible polymeric material to a skin wound, abrasion, burn or other skin injury, in which the solution may contain one or more skin-irritating chemicals, such as diethyl ether, ethanol, ethyl acetate, methyl acetate, acetone, methyl ethyl ketone and combinations thereof. The method includes: (i) providing a composition containing between 1% and 10% by (dry) weight of the skin-compatible polymeric material dissolved in a liquid medium suitable for human topical use, in which the liquid medium contains less than 20% (combined weight percent) of one or more skin-irritating chemicals such as the above, at least 45% (combined weight percent) of at least one volatile solvent and/or volatile co-solvent, in which the molecules constituting the stated 45% solvent and/or co-solvent contain at least 5 carbon atoms, and at least 20% by weight of at least one volatile hydrocarbon diluent liquid. The diluent liquid has an initial boiling point at 1 atmosphere of greater than 35° C. and less than 150° C., and the liquid medium causes little or no stinging when applied to a skin injury, and (ii) applying the composition to the skin of a patient at the site of the skin injury.

In one embodiment of this aspect, the skin-compatible polymeric material is nitrocellulose.

In another embodiment, the skin-compatible polymeric material is an acrylate-containing polymer or co-polymer such as poly(methylacrylate-isobutene-monoisopropylmaleate.

In still another embodiment, the volatile hydrocarbon diluent liquid includes at least one aliphatic hydrocarbon liquid.

In a related embodiment, the aliphatic hydrocarbon liquid is an alkane liquid.

In another related embodiment, the alkane liquid is selected from the group consisting of n-hexane, structural isomers of n-hexane and combinations thereof.

In yet another related embodiment, the alkane is selected from the group consisting of n-heptane, structural isomers of n-heptane and combinations thereof.

In still another related embodiment, the alkane is selected from the group consisting of n-pentane, n-octane, structural isomers of n-pentane, n-octane and combinations thereof.

In another related embodiment, the alkane is isohexane.

In another aspect, the invention features a composition containing between 1% and 10% by (dry) weight of a skin-compatible polymeric material dissolved in a liquid medium suitable for human topical use, in which the liquid medium contains less than 20% (combined weight percent) of one or more skin-irritating chemicals, such as diethyl ether, ethanol, ethyl acetate, methyl acetate, acetone, methyl ethyl ketone and combinations thereof that cause stinging when applied to a skin wound, abrasion, burn or other skin injury, and in which the liquid medium further contains at least 70% (combined weight percent) of at least one volatile solvent and/or volatile co-solvent, and optional volatile diluent(s) in which the molecules constituting the stated solvent and/or co-solvent contain at least 5 carbon atoms, and the liquid medium has an initial boiling point at 1 atmosphere of greater than 35° C. and less than 150° C. The liquid medium causes little or no stinging when applied to a skin injury.

In one embodiment of this aspect, the skin-compatible polymeric material is nitrocellulose.

In another embodiment, the skin-compatible polymeric material is an acrylate-containing polymer or co-polymer such as poly(methylacrylate-isobutene-monoisopropylmaleate.

In still another embodiment, at least one volatile solvent in the composition is an acetate solvent.

In a related embodiment, the acetate solvent is an alkyl acetate solvent.

In another related embodiment, the alkyl acetate solvent is selected from the group consisting of n-propyl acetate, n-butyl acetate, n-amyl acetate, structural isomers of n-propyl acetate, n-butyl acetate and n-amyl acetate, and combinations thereof.

In yet another related embodiment, the alkyl acetate solvent is selected from the group consisting of n-butyl acetate, n-amyl acetate, structural isomers of n-butyl acetate and n-amyl acetate, and combinations thereof.

In another embodiment, the liquid medium further includes at least one ingredient selected from the group consisting of co-solvent, humectants, emollients, antiseptic agents, and combinations thereof.

In a related embodiment, at least one ingredient is selected from the group consisting of methyl propanediol, castor oil, benzalkonium chloride and combinations thereof.

In another embodiment, the diluent liquid is a volatile alkane liquid or combination of volatile alkane liquids.

In a related embodiment, the volatile alkane liquid is selected from the group consisting of n-hexane, structural isomers of n-hexane and combinations thereof.

In another related embodiment, the volatile alkane liquid is selected from the group consisting of n-heptane, structural isomers of n-heptane and combinations thereof.

In another related embodiment, the volatile alkane liquid is selected from the group consisting of n-pentane, n-octane, structural isomers of n-pentane and n-octane and combinations thereof.

In another related embodiment, the volatile alkane liquid is isohexane.

In yet other related embodiments, the liquid medium contains greater than 75% or even greater than 85% (combined weight percent) of at least one volatile solvent and/or volatile co-solvent, and optional volatile diluent(s).

The following examples are intended to further illustrate, but not limit, the invention.

### EXAMPLES

### Example 1

### Prior Art Collodion Formulation Used as First Aid Antiseptic Liquid Bandage

The New-Skin™ liquid bandage product (Medtech, Inc.) is herein described and provided as an example of a commercial collodion preparation that has served to treat minor skin injuries such as small cuts. The manufacturer states, “New-Skin dries rapidly to form a tough protective cover that is antiseptic, flexible, waterproof and lets the skin breathe. Completely covers the entire wound to keep out dirt and germs.” Its uses are listed as follows: “Protects cuts and scrapes. Prevents and protects blisters. Helps prevent the formation of calluses. Covers painful hangnails. Particularly useful for bowlers, golfers, tennis players, fishermen and musicians.” Its directions for use are as follows: “Clean affected area. Apply a small amount on the area 1-3 times daily. Let dry. A second coating may be applied for extra protection. To remove, apply more New-Skin and quickly wipe off. Fingernail polish remover may dissolve New-Skin.”

The manufacturer has disclosed to Applicant that, among other proprietary ingredients, the current New-Skin collodion formulation contains approximately 40% by weight ethyl acetate and 8% alcohol (ethanol+isopropanol) as well as approximately 24% butyl acetate and 18% amyl acetate. Applicant has now determined that the ethyl acetate and the alcohol are largely responsible for the stinging and burning that is experienced when New-Skin is applied to an open cut or fresh skin abrasion (see sting test results above). It is noted that this formulation includes two solvents that do not cause stinging (butyl and amyl acetate totaling approximately 42% by weight). However, these ingredients provided no obvious or discernable benefit in terms of sting relief because of the overwhelming stinging caused by ethyl acetate and alcohol as shown by Applicant. The level of stinging produced by New-Skin (as scored by sensory criteria described earlier) is +++.

### Example 2

### Acetate-Alkane Solvent System Used to Formulate “No-Sting” Collodion Liquid Bandage

As described previously herein, higher molecular weight alkyl acetates (i.e., alkyl esters of acetic acid) such as butyl acetate and amyl acetate, as well as the alkanes such as hexane and heptane, produce little or no stinging when applied to abraded skin. However, because n-butyl acetate evaporates 6-7 fold more slowly at room temperature than ethyl acetate (abundant in the original formulation of New-Skin), a purely butyl acetate solvent-containing collodion would dry too slowly. Therefore, Applicant blended a more volatile alkane solvent with the less volatile butyl acetate to arrive at a solvent blend that would allow the collodion to thicken and dry within an acceptable period of time. For example, n-hexane is useful because it evaporates approximately ten-fold more rapidly than n-butyl acetate and approximately 1.5 times more rapidly than ethyl acetate. N-heptane also evaporates rapidly, but not quite as than ethyl acetate (about 0.6 times as rapidly). On the other hand, the alkanes are co-solvents rather than true solvents for the nitrocellulose present in collodion preparations. Therefore, there is a limit to the proportion of an alkane that can be incorporated into a solvent blend for a collodion composition without causing precipitation of the nitrocellulose. Applicant observed that the amount of alkane such as n-hexane, n-heptane, and isomers thereof in an acetate solvent-based collodion preparation is preferably maintained below 40% by weight to avoid precipitation. For example, 34-38 parts by weight n-hexane or n-heptane and/or their isomers could be combined with approximately 53-58 parts of n-butyl acetate and/or its isomers, and also combined with approximately 5 parts nitrocellulose (a range of 3 to 7 parts as calculated on dry weight basis) and optionally approximately 0-6 parts of various additives including emollients, humectants and plasticizers. Accordingly, the following collodion formulation was prepared:

| Formulation #1: By Weight | Functionality |
|---|---|
| 56.0% n-butyl acetate CAS# 123-86-4 | solvent |
| 34.4% n-hexane CAS# 110-54-3 | diluent |
| 5.5% wet nitrocellulose CAS# 9004-70-0 | film former |
| type RS 60-80 sec (containing 3.85% dry | |
| nitrocellulose + 1.65% isopropanol) | |
| 2.0% castor oil CAS# 8001-79-4 | plasticizer, emollient |
| 2.0% methyl propanediol CAS# 2163-42-0 | humectant |
| 0.10% benzalkonium chloride CAS# 68391-01-5 | topical antiseptic |

After the ingredients were weighed out, the nitrocellulose (70% dry weight: 30% isopropanol) was saturated with n-hexane. Next, n-butyl acetate was added and the mixture was subjected to high shear mixing and incubation overnight at room temperature until the nitrocellulose had completely dissolved. The remaining ingredients were added and thoroughly mixed together. Three derivatives of Formulation #1 were subsequently prepared using the same procedure, but with several additional ingredients:

#2: 100 parts by weight formulation #1 plus 2 parts benzyl alcohol CAS# 100-51-6 (plasticizer),

#3: 100 parts by weight formulation #1 plus 1 part emollient, e.g., triglyceride mixture,

#4: 100 parts by weight formulation #1 plus 2 parts benzyl alcohol plus 1 part emollient, e.g., triglyceride mixture.

Using the Standard Sting Test, all four of the above formulations scored either − or +/− and were deemed satisfactory. Formulation #4 provides both an additional emollient and a film plasticizer.

### Example 3

### Different Diluents Affect Drying Time of Butyl Acetate Solvent-Based “No-Sting” Collodions

The n-butyl acetate solvent-based collodion formulation described in Example 2 was modified by replacing n-hexane with the same amount of either a faster evaporating or a slower evaporating alkane diluent. The purpose of this experiment was to determine whether the choice of diluent would significantly alter the collodion drying time or the performance of the final dried film. In one instance, the somewhat faster evaporating isomer of n-hexane, isohexane (principally 2-methylpentane, CAS# 107-83-5) was substituted for n-hexane. Isohexane has an initial boiling point of approximately 60° C. (manufactured by South Hampton Refining Co., Silsbee, Tex.). In the other instance, n-heptane (CAS# 142-82-5) with a boiling point of 98° C. was substituted. These compare with n-hexane having a boiling point of 69° C. Both formulations were prepared as described in Example 2. The formulations produced clear collodion solutions that, when applied by brush to human skin and to a hard surface (glass), dried to form clear films. These films were equivalent to the film produced using n-hexane. However, there was a moderate difference in the rate of drying at human skin temperature (37° C.). Drying rates (measured by solvent weight loss from identical size samples) showed that the isohexane formulation dried approximately 10% more rapidly, and the heptane dried approximately 10% more slowly than both the n-hexane-containing formulation in Example 2 and the original New Skin formulation. The latter two formulations dried at comparable rates.

Unless otherwise defined herein, all terms have their ordinary meanings as understood by one of ordinary skill in the field to which the invention pertains. All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, collodion compositions that are constituted using other non-stinging solvents, latent solvents, co-solvents and diluents not listed herein, or equivalent collodion coatings incorporated into topical products not listed herein, are all within the scope of the present invention. Thus, such additional embodiments are within the scope of the present invention and the following claims.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any two different values as the endpoints of a range. Such ranges are also within the scope of the described invention.

Thus, additional embodiments are within the scope of the invention and within the following claims.

1. A method of reducing or eliminating stinging that may accompany application of a topical solution of a skin-compatible polymeric material to a skin wound, abrasion, burn or other skin injury, wherein said method comprises:
(i) providing a composition comprising between 1% and 10% by weight of said polymeric material dissolved in a liquid medium suitable for human topical use, wherein said liquid medium comprises less than 20% combined weight of one or more skin-irritating chemicals, and at least 45% combined weight of at least one volatile solvent and/or volatile co-solvent, wherein the molecules constituting said at least one volatile solvent or volatile co-solvent contain at least 5 carbon atoms, and wherein said liquid medium has an initial boiling point at 1 atmosphere of greater than 35° C. and less than 150° C., and causes little or no stinging when applied to a skin injury; and
(ii) applying said composition to the skin of a patient at said skin injury.

2. The method of claim 1, wherein, in said composition, said one or more skin-irritating chemicals are selected from the group consisting of diethyl ether, ethanol, ethyl acetate, methyl acetate, acetone and methyl ethyl ketone.

3. The method of claim 1, wherein, in said composition, said liquid medium comprises less than 10% of said one or more skin-irritating chemicals.

4. The method of claim 1, wherein, in said composition, said liquid medium comprises less than 5% of said one or more skin-irritating chemicals.

5. The method of claim 1, wherein, in said composition, said liquid medium comprises greater than 65% of said at least one volatile solvent and/or volatile co-solvent.

6. The method of claim 1, wherein, in said composition, said liquid medium comprises greater than 85% of said at least one volatile solvent and/or volatile co-solvent.

7. The method of claim 1 wherein, in said composition, said polymeric material is nitrocellulose.

8. The method of claim 1 wherein, in said composition, said polymeric material is an acrylate-containing polymer or co-polymer.

9. The method of claim 1 wherein, in said composition, said volatile solvent or co-solvent is an acetate solvent.

10. The method of claim 9 wherein, in said composition, said acetate solvent is an alkyl acetate solvent selected from the group consisting of n-propyl acetate, n-butyl acetate, n-amyl acetate, structural isomers of n-propyl acetate, n-butyl acetate and n-amyl acetate, and combinations thereof.

11. The method of claim 9 wherein, in said composition, said acetate solvent is an alkyl acetate solvent selected from the group consisting of n-butyl acetate, n-amyl acetate, structural isomers of n-butyl acetate and n-amyl acetate, and combinations thereof.

12. The method of claim 7 wherein, in said composition, said nitrocellulose solution composition is selected from the group consisting of collodion, flexible collodion, styptic collodion, cantharidal collodion, and any other nitrocellulose-based solution composition that is supplemented with at least one topically active pharmacological agent.

13. The method of claim 12 wherein, in said composition, said pharmacological agent is selected from the group consisting of hormones, local anesthetics, keratolytic agents, ultraviolet light protective agents, and combinations thereof.

14. The method of claim 13 wherein, in said composition, a hormone is selected from the group consisting of steroids, sexual hormones, and metabolic regulatory hormones.

15. A method of reducing or eliminating stinging that may accompany application of a topical solution of a skin-compatible polymeric material to a skin wound, abrasion, burn or other skin injury, wherein said method comprises:
(i) providing a composition comprising between 1% and 10% by weight of said polymeric material dissolved in a liquid medium suitable for human topical use, wherein said liquid medium comprises less than 20% combined weight of one or more skin-irritating chemicals, at least 45% combined weight of at least one volatile solvent and/or volatile co-solvent, wherein the molecules constituting said at least one volatile solvent and/or volatile co-solvent contain at least 5 carbon atoms, and at least 20% by weight of at least one volatile hydrocarbon diluent liquid, and wherein said liquid medium has an initial boiling point at 1 atmosphere of greater than 35° C. and less than 150° C., and causes little or no stinging when applied to a skin injury,
(ii) applying said composition to the skin of a patient at said skin injury.

16. The method of claim 15, wherein, in said composition, said one or more skin-irritating chemicals are selected from the group consisting of diethyl ether, ethanol, ethyl acetate, methyl acetate, acetone and methyl ethyl ketone.

17. The method of claim 15, wherein, in said composition, said liquid medium comprises less than 10% of said one or more skin-irritating chemicals.

18. The method of claim 15, wherein, in said composition, said liquid medium comprises less than 5% of said one or more skin-irritating chemicals.

19. The method of claim 15, wherein, in said composition, said liquid medium comprises greater than 65% of said at least one volatile solvent and/or volatile co-solvent.

20. The method of claim 15, wherein, in said composition, said liquid medium comprises greater than 85% of said at least one volatile solvent and/or volatile co-solvent.

21. The method of claim 15 wherein, in said composition, said volatile hydrocarbon diluent liquid comprises at least one aliphatic hydrocarbon liquid.

22. The method of claim 21 wherein, in said composition, said at least one aliphatic hydrocarbon liquid is an alkane liquid.

23. The method of claim 22 wherein, in said composition, said alkane liquid is selected from the group consisting of n-hexane, structural isomers of n-hexane and combinations thereof.

24. The method of claim 22 wherein, in said composition, said alkane is selected from the group consisting of n-heptane, structural isomers of n-heptane and combinations thereof.

25. The method of claim 22 wherein, in said composition, said alkane is selected from the group consisting of n-pentane, n-octane, structural isomers of n-pentane and n-octane and combinations thereof.

26. The method of claim 22 wherein, in said composition, said alkane is isohexane.

27. A composition comprising between 1% and 10% by weight of a skin-compatible polymeric material dissolved in a liquid medium suitable for human topical use, wherein said liquid medium contains less than 20% combined weight of one or more skin-irritating chemicals and at least 70% combined weight of at least one volatile solvent and/or volatile co-solvent, and optional volatile diluent(s) wherein the molecules constituting said at least one volatile solvent and/or volatile co-solvent contain at least 5 carbon atoms, and wherein said liquid medium has an initial boiling point at 1 atmosphere of greater than 35° C. and less than 150° C., and causes little or no stinging when applied to a skin injury.

28. The composition of claim 27, wherein said one or more skin-irritating chemicals are selected from the group consisting of diethyl ether, ethanol, ethyl acetate, methyl acetate, acetone and methyl ethyl ketone.

29. The composition of claim 27, wherein said liquid medium comprises less than 10% of said one or more skin-irritating chemicals.

30. The composition of claim 27, wherein said liquid medium comprises less than 5% of said one or more skin-irritating chemicals.

31. The composition of claim 27, wherein said liquid medium comprises greater than 75% of said at least one volatile solvent and/or volatile co-solvent, and optional volatile diluent(s).

32. The composition of claim 27, wherein said liquid medium comprises greater than 85% of said at least one volatile solvent and/or volatile co-solvent, and optional volatile diluent(s).

33. The composition of claim 27 wherein said polymeric material is nitrocellulose.

34. The composition of claim 27 wherein said polymeric material is an acrylate-containing polymer or co-polymer material.

35. The composition of claim 27 wherein said volatile solvent and/or co-solvent is an acetate solvent.

36. The composition of claim 35 wherein said acetate solvent is an alkyl acetate solvent.

37. The composition of claim 36 wherein said alkyl acetate solvent is selected from the group consisting of n-propyl acetate, n-butyl acetate, n-amyl acetate, structural isomers of n-propyl acetate, n-butyl acetate and n-amyl acetate, and combinations thereof.

38. The composition of claim 36 wherein said alkyl acetate solvent is selected from the group consisting of n-butyl acetate, n-amyl acetate, structural isomers of n-butyl acetate and n-amyl acetate, and combinations thereof.

39. The composition of claim 27 wherein said liquid medium further comprises at least one ingredient selected from the group consisting of co-solvents, humectants, emollients, antiseptic agents, and combinations thereof.

40. The composition of claim 39 wherein said at least one ingredient is selected from the group consisting of methyl propanediol, castor oil, benzalkonium chloride and combinations thereof.

41. The composition of claim 27 wherein said diluent liquid is a volatile alkane liquid or combination of volatile alkane liquids.

42. The composition of claim 41 wherein said volatile alkane liquid is selected from the group consisting of n-hexane, structural isomers of n-hexane and combinations thereof.

43. The composition of claim 41 wherein said volatile alkane liquid is selected from the group consisting of n-heptane, structural isomers of n-heptane and combinations thereof.

44. The composition of claim 41 wherein said volatile alkane liquid is selected from the group consisting of n-pentane, n-octane, structural isomers of n-pentane and n-octane and combinations thereof.

45. The composition of claim 41 wherein said volatile alkane liquid is isohexane.
